(19) United States
(12) Patent Application Publication
(10) Pub. No.: US 2015/0238747 A1
(43) Pub. Date: Aug. 27, 2015

(54) CONNECTOR WITH NON-LUER COMPATIBLE ACCESS PORTS'

(71) Applicant: Dale Medical Products, Inc., Plainville, MA (US)

(72) Inventor: Ronald D. RUSSO, Naples, FL (US)

(73) Assignee: DALE MEDICAL PRODUCTS, INC.

(21) Appl. No.: 14/630,160

(22) Filed: Feb. 24, 2015

Related U.S. Application Data
(60) Provisional application No. 61/966,383, filed on Feb. 24, 2014.

(51) Int. Cl. A61M 39/10

(52) U.S. Cl. CPC A61M 39/1011

(57) ABSTRACT

The present disclosure provides a connector preventing use and mechanical engagement with an I.V. luer catheter having luer compatible fittings. The connector comprises a body portion having a non-luer compatible distal patient access fitting, a non-luer compatible proximal fluid delivery device fitting, and a non-luer syringe port positioned between the distal and proximal non-luer compatible fittings. In some embodiments, the syringe port incorporates a normally closed to atmosphere valve accessible by a non-luer syringe for administration of fluid and medications.

### PRIORITY

This application claims priority under 35 U.S.C. §119 to U.S. Provisional Patent Application No. 61/966,383, filed on Feb. 24, 2014, which is herein incorporated in its entirety by reference.

### TECHNICAL FIELD

The present disclosure relates to medical connector devices for enteral applications.

### BACKGROUND

The present disclosure relates to medical connector devices for enteral applications. The devices can be used to connect non-luer catheters and non-luer syringes, such as enteral catheters and enteral irrigation syringes.

Intravenous (“I.V.”) catheters are typically used to administer I.V. medications into a patient's I.V. or arterial system using catheters having luer fittings defined by ISO 594:1986 “Conical fittings with a 6% (Luer) taper for syringes, needles and certain other medical equipment” standards (“ISO/ANSI 1986”). An ISO/ANSI 1986 compliant, or luer catheter typically has a female rigid luer fitting which will fit into a male luer I.V. syringe or I.V. extension or I.V. administration set. From the luer dimensional ISO/ANSI 1986 standards, the luer tip has an outside diameter of 4 mm, a flow path of less than 2.4 mm in diameter, and a length of less than 0.300 inches with a 6% taper.

By comparison, enteral catheters or tubes typically have randomly configured access ports, some of which may have luer-compatible access ports. As such, clinicians may inadvertently use I.V. luer tip or luer lock syringes to connect to an enteral catheter that may be I.V. luer-compatible. But, the administration of enteral formula into a patient's I.V. system can have catastrophic effects leading to serious complications and/or death.

Manufacturers have produced products which try to prevent this occurrence by providing enteral connectors which attempt to prevent connection to rigid I.V. luer ports. Examples include the Lopez® Valve marketed by ICU Medical Inc., and the ACE Connector® marketed by Dale® Medical Products, Inc. The Dale ACE Connector® is especially useful in preventing misconnection to I.V. luer syringe tips in that it utilizes embodiments of a syringe port seal as shown and described in U.S. Pat. No. 8,353,895 issued on Jan. 15, 2013 to Russo. The syringe port seal of Dale Medical Products, Inc. described in U.S. Pat. No. 8,353,895 will only accept a randomly dimensioned non-luer tip syringe typically referred to as a catheter tip syringe. It should be noted that the term “catheter tip syringe,” as used herein refers to any tapered tip syringe having dimensions, e.g., tip diameter and tip length, in excess of the ISO/ANSI 1986 standard.

While the Dale ACE Connector® is a substantial advancement in the state of the art, ISO and ANSI have gone further in proposing standards for non-luer compatible fittings of all syringes, feeding tubes, and delivery sets having small bore connector fittings with less than 8.5 mm in diameter flow path for enteral administration. These standards have been issued as proposed ISO/ANSI 80369-3 “Small-bore connectors for liquids and gases in healthcare applications—part 3: connectors for enteral applications” standards (“ISO/ANSI 80369-3”) for syringes, feeding tubes, and delivery sets having totally non-luer compatible male and female oversized screw thread type fittings.

U.S. Pat. No. 5,267,983 to Oilschlager et. al. (“Oilschlager”) titled “Enteral Adapter and Tip Protector” issued on Dec. 7, 1993, discloses an adapter for an enteral feed tube. The adapter is a multi-step adapter, where the first step is taught to be less than 0.170 inches (4.3 mm). Similarly, U.S. Pat. No. 5,776,117 to Haselhorst et. al. (“Haselhorst”) titled “Adaptor for Connection to Variously Sized Tubes, Adapters and/or Y-Ports and Method of Using the Adapter,” issued Jul. 7, 1998, also discloses an enteral adapter having a series of increased diameter barbed sections for insertion into a wide variety of varying internal diameter tubes, adapters, or Y ports. The Haselhorst adapter has a first step of 0.180 inches (4.57 mm).

### SUMMARY

The inventor has recognized that it would be especially useful to have a closed system enteral connector having multiple access connection ports wherein all access or connection ports are totally incompatible with any type of luer syringe or I.V. luer fluid delivery set, such that all ports are configured to prevent use, attachment, or mechanical engagement with any luer fitting. This configuration, the inventor has recognized, would absolutely prevent the administration of any enteral formula into any type of I.V. luer catheter or fitting whether rigid or flexibly molded.

The inventor has further recognized that the Oilschlager configuration, having a multi-step adapter, where the first step is less than 0.170 inches (4.3 mm), still permits a perfect diameter fit with the internal diameter of a standard female I.V. luer port. Similarly, Haselhorst, which does not recognize the importance of any such dimensions relative to preventing connection to any I.V. luer fitting, specifies that the first step has a dimension of 0.180 inches (5.47 mm). To overcome the I.V. luer misconnection possibility, Oilschlager depends upon a shortened length of the first step of only 0.140 inches (3.6 mm) to prevent a sustained internal grip with the I.V. luer port. In essence, the adapter of Oilschlager (and Haselhorst) provide for a partial insertion compatibility fit with an I.V. luer port, and although the connection may not be sustained, the connection can still permit the administration of enteral formula into an I.V. luer port. FIG. 8 in Oilschlager depicts this scenario. Moreover, a more substantial insertion of Oilschlager or Haselhorst's adapters is possible if the I.V. luer port is molded out of flexible walled PVC plastic approximately 65 shore A durometer, which is often the case. Specifically, the first section step dimensions utilized in the commercial embodiments of both Oilschlager and Haselhorst may permit both adapters to be connected to the flexible PVC plastic molded female luer fluid irrigation administration ports on tracheal suction patient airway catheters, for example. Further, since both Olischlager and Haselhorst are single port enteral adapters they are of little use in the conception of a multi-ported closed system enteral connector wherein all ports have varying functions and operations.

In one embodiment of the present disclosure, a medical connector prevents use and mechanical engagement with an I.V. luer catheter, the connector comprising a body portion having a distal patient access fitting having a first non-luer compatible form, a proximal fluid delivery device fitting having a second non-luer compatible form, and a syringe port incorporating a normally biased closed to atmosphere valve, the syringe port having a third non-luer compatible form, the third non-luer compatible form comprising a vertical length greater than about 0.300 inches from the entrance of the syringe port to the valve, wherein the distal patient access fitting, the proximal fluid delivery device fitting, and the syringe port are all in fluid communication with a fluid flow path positioned within the body portion.

In some embodiments, the first non-luer compatible form and the second non-luer compatible form are the same form, while in others they may take different forms. The first and second non-luer compatible form may be, in some embodiments, selected from the group consisting of a female externally threaded form having an internal diameter of greater than 4.8 mm, a stepped barb form comprising at least a first stepped end barb having a first diameter of greater than about 0.190 inches (4.8 mm), and a second stepped barb having a second diameter greater than the first diameter, and a male stem form having a diameter of greater than about 0.190 inches (4.8 mm). In other embodiments, the diameters are greater than about 0.205 inches (5.2 mm).

In yet another embodiment, the medical connector has an adapter engaged with the syringe port, wherein the adapter mechanically engages with an enteral fluid delivery device. The adapter may access the normally biased closed to atmosphere valve, causing the valve to be in an open position permitting fluid flow from the enteral fluid delivery device into the fluid flow path positioned within the body portion. Further, the body portion may have an On-Off fluid flow controller disposed between the valve and the proximal fluid delivery device fitting, which may also have a manually operated turn handle. In some embodiments, the turn handle may have fixed stop positions for both the on and off fluid flow positions. In some embodiments, the valved syringe access port may accept a catheter tip syringe having a length in excess of about 0.300 inches. And, in some embodiments, the distal patient access fitting has a flow path internal diameter dimensionally in excess of 2.4 mm, and further, the proximal fluid delivery device fitting may also have a flow path internal diameter dimensionally in excess of 2.4 mm.

A connector consistent with the present disclosure may have an adapter attached to the distal patient access fitting, permitting connection of the device to an enteral feeding tube, and in some embodiments, the adapter may have a non-luer compatible form comprising a male stem having a tapered outside diameter of greater than about 4.8 mm, or in other embodiments greater than about 5.2 mm.

In still other embodiments, the proximal fluid delivery device fitting connects to an enteral feeding device. The connector may be an enteral formula delivery connector.

In some embodiments, the proximal fluid delivery device fitting may be removable from the body portion, and the removal of the proximal fluid delivery device fitting may expose an underlying auxiliary suction line connector part of the body portion.

Also consistent with the present disclosure, a medical connector kit may prevent the use and mechanical engagement with an I.V. luer catheter, the connector kit comprising a medical connector comprising a body portion having a distal patient access fitting having a first non-luer compatible form, a proximal fluid delivery device fitting having a second non-luer compatible form, and a syringe port incorporating a normally biased closed to atmosphere valve, the syringe port having a third non-luer compatible form, the third non-luer compatible form comprising a vertical length greater than about 0.300 inches from the entrance of the syringe port to the valve, wherein the distal patient access fitting, the proximal fluid delivery device fitting, and the syringe port are all in fluid communication with a fluid flow path positioned within the body portion; and at least one adapter. The at least one adapter may comprise a proximal portion comprising a taper lock wedge, the taper lock wedge having a minimum diameter in excess of about 4.8 mm, or in other embodiments about 5.2 mm, and a rearward portion comprising molded-in threads. In some embodiments, the taper lock wedge of the proximal portion of the adapter may have a vertical length greater than about 0.300 inches.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

The accompanying drawings, which are incorporated in and constitute a part of this specification, illustrate embodiments of the invention and, together with the description, explain the principles of the invention. In the drawings:

FIG. 1 is a partial cross sectional view of an exemplary syringe port seal consistent with the disclosure in U.S. Pat. No. 8,353,895.

FIG. 2 is a partial cross sectional view of an exemplary syringe port seal consistent with the disclosure as in U.S. Pat. No. 8,353,895, depicting a non-luer compatible catheter tip syringe accessing the syringe port seal.

FIG. 3 is a partial cross sectional view of an exemplary syringe port seal consistent with the disclosure as in U.S. Pat. No. 8,353,895, wherein a male luer slip tip syringe is prevented from accessing the syringe port seal.

FIG. 4 is a partial cross sectional view of an exemplary syringe port seal consistent with the disclosure as in U.S. Pat. No. 8,353,895, wherein a male luer lock tip syringe is prevented from accessing the syringe port seal.

FIG. 5 is a top view of a first exemplary embodiment of the connector depicting all access ports not capable of use, attachment or engagement with luer tip devices.

FIG. 6 is a partial cross sectional view of the exemplary non-luer compatible syringe port seal incorporated as part of the connector depicted in FIG. 5.

FIG. 7 is a side view of the exemplary connector depicted in FIG. 5 wherein the distal patient access fitting is directly attached to an enteral feeding tube such as a gastrostomy tube.

FIG. 8 is a top view of a second exemplary embodiment of the connector wherein the distal patient access fitting is a dimensionally oversized externally threaded female fitting preventing use, attachment, or mechanical engagement with an I.V. luer catheter in a patient's arm.

FIG. 9 is a side view of the second exemplary embodiment of the connector from FIG. 8.

FIG. 10 is a cross sectional side view of the second exemplary embodiment of the connector depicting all ports in fluid flow communication with a fluid flow path wherein the distal patient access fitting is totally incompatible with an I.V. luer catheter in a patient's arm.

FIG. 11 is the same second exemplary embodiment of the connector from FIG. 10 wherein the proximal fluid delivery device fitting is totally incompatible with an I.V. luer catheter in a patient's arm.

FIG. 12 is the same second exemplary embodiment of the connector from FIG. 10 depicting the removal of proximal fluid delivery device fitting.

FIG. 13 is the same second exemplary embodiment of the connector from FIG. 12 depicting the proximal fitting removed from the body of the connector exposing an underlying auxiliary suction line connector part of the body portion.

FIG. 14 is an exemplary packaged version of the second exemplary embodiment of the connector from FIG. 10 wherein the package includes two adapters providing further non-luer attachment capabilities to both the distal fitting and the syringe port seal.

FIG. 15 is the exemplary adapter from FIG. 14 packaged separately as a replacement adapter for use with the exemplary connector depicted in FIG. 14.

FIG. 16 is a partial cross sectional side view of the second exemplary embodiment of the connector from FIG. 10 depicting all the various non-luer compatible devices that can be used, attached, or mechanically engaged with the connector without the use of the exemplary adapters depicted in FIG. 14.

FIG. 17 is a partial cross section side view of the second exemplary embodiment of the connector from FIG. 10 depicting all the various non-luer compatible devices that can be used, attached, or mechanically engaged with the connector while utilizing the exemplary adapters depicted in FIG. 14.

### DESCRIPTION OF EXEMPLARY EMBODIMENTS

Reference will now be made in detail to the present exemplary embodiments, examples of which are illustrated in the accompanying drawings. Wherever possible, the same reference numbers will be used throughout the drawings to refer to the same or like parts.

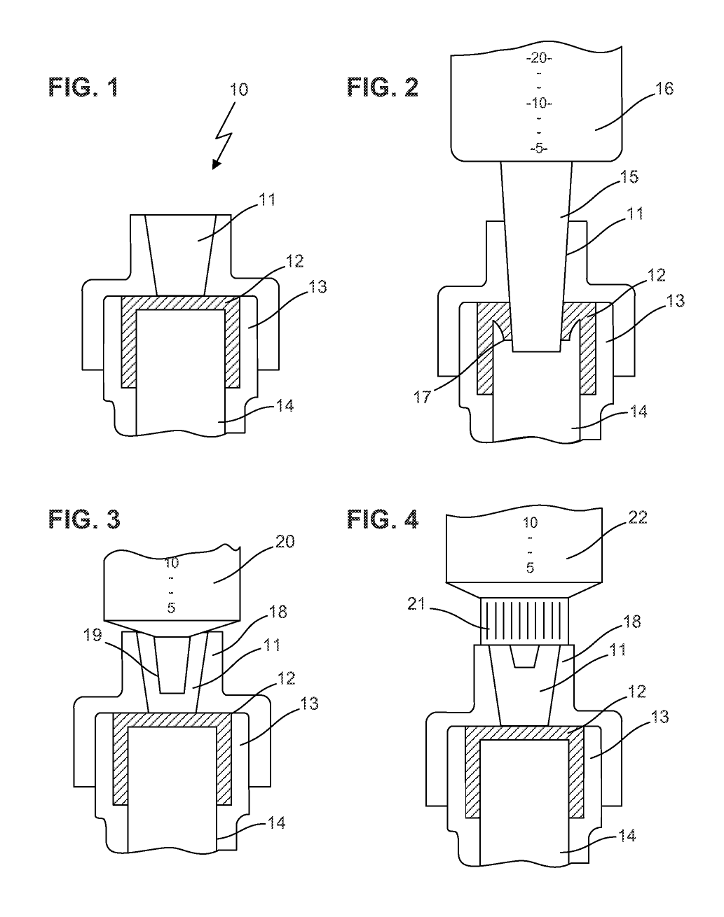

FIGS. 1-4 depict of an exemplary syringe port seal consistent with the disclosure as in U.S. Pat. No. 8,353,895.

FIG. 1 depicts a partial cross section of the exemplary syringe port seal 10 having a plastic upper entrance port 11 with an internal silicone slit valve 12 fitted into housing 13 communicating with lower fluid path 14. FIG. 2 depicts that the upper entrance port 11 forms a tapered wedge fit with a tapered nozzle non-luer compatible tip 15 on a catheter tip syringe 16, where the tip 15 purses open slit 17 on valve 12 to permit fluid flow from syringe 16 into lower fluid path 14.

From the luer dimensional ISO/ANSI 1986 standards, the luer tip has an outside diameter of 4 mm, a fluid flow path of less than 2.4 mm in diameter, and a length of less than 0.300 inches with a 6% taper. The length and outside diameter of tip 15 on catheter tip syringe 16 is so oversized in dimensions (having length greater than about 0.300 inches, and diameter greater than 4.8 mm) such that any luer slip tip or any luer lock syringe simply cannot access entrance port 11 as illustrated in FIGS. 3 and 4.

FIG. 3 depicts entrance port 11 having a length in excess of 0.300 inches and an opening 18 of approximately 8.0 mm in internal diameter, which prevents any luer slip tip 19 on a luer tip syringe 20 from accessing and engaging with entrance port 11 and preventing tip 19 from opening valve 12, which remains closed. The same is true for any luer lock syringe tip 21 on luer lock syringe 22 depicted in FIG. 4.

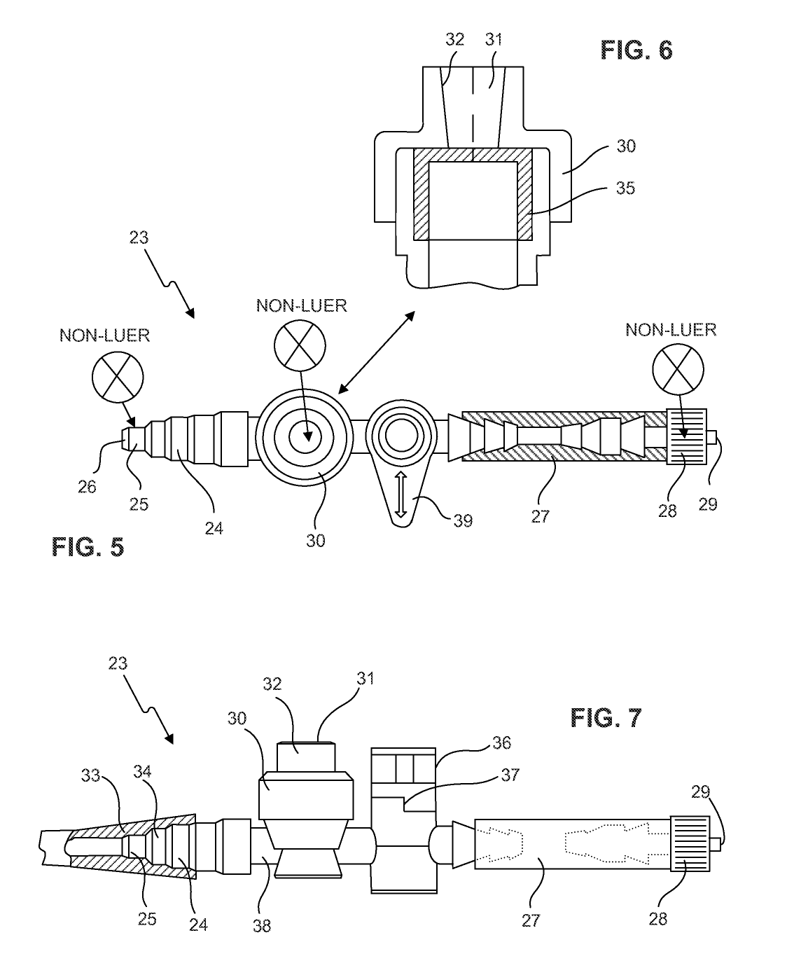

FIG. 5 is a top view of the first exemplary embodiment of closed system triple ported connector 23 being an enteral formula delivery connector having a non-luer compatible distal patient access fitting 24 wherein the distal first stepped end 25 is oversized in dimension with a diameter of 5.2 mm and a fluid flow path 26 of 3.14 mm. As such, fitting 24 is configured to prevent use, attachment, or engagement with any female I.V. luer fitting, which has a much smaller diameter of 4 mm. As such, the oversized 5.2 mm distal first stepped end 25 on fitting 24 simply cannot be inserted into the much smaller 4 mm internal diameter of a female luer fitting. Distal patient access fitting 24 is configured as a male tapered stepped barbed tube fitting permitting attachment to a wide variety of enteral feeding tubes.

As used herein, a stepped end barb refers to the first step in the barbed fitting, the end-most and typically smallest barb. Distal first stepped end 25 with the oversized diameter of about 5.2 mm (0.205 inches) means that distal first stepped end 25 will butt up against the smaller I.V. luer lumen of about 4 mm (0.158 inches). As such, distal first stepped end 25 simply cannot even begin to enter or form any type of partial or limited insertion fit with an I.V. luer port. Stepped barbs are particularly useful to accommodate various sized gastrostomy tubes, and a diameter of about 0.205 inches is small enough to accommodate the smallest adult gastrostomy tube of 18 FR while still preventing any type of connection with an I.V. luer fitting.

Likewise, in FIG. 5 connector 23 has a non-luer compatible proximal fluid delivery fitting 27 which incorporates male stepped barbed port 28 with a male stem 29. The male stem 29 has a tapered outside diameter of 5.7 mm, which again is so oversized that it too cannot be used, attached, or mechanically engaged with any female I.V. luer fitting of only 4 mm in internal diameter. Further, the male stem 29 part of fitting 27 prevents any type of mating compatibility, engagement, or physical connection with any type of male luer fitting used on any type of I.V. administration set.

FIG. 5 also clearly depicts that in the exemplary embodiment of the closed system triple-ported connector 23, syringe port seal 30 is also configured to prevent use, attachment, or mechanical engagement with any luer tip syringe as previously shown and described in FIGS. 3 and 4. The syringe port seal 30 provides an in-line means to deliver fluids and/or medications without disconnecting the catheter.

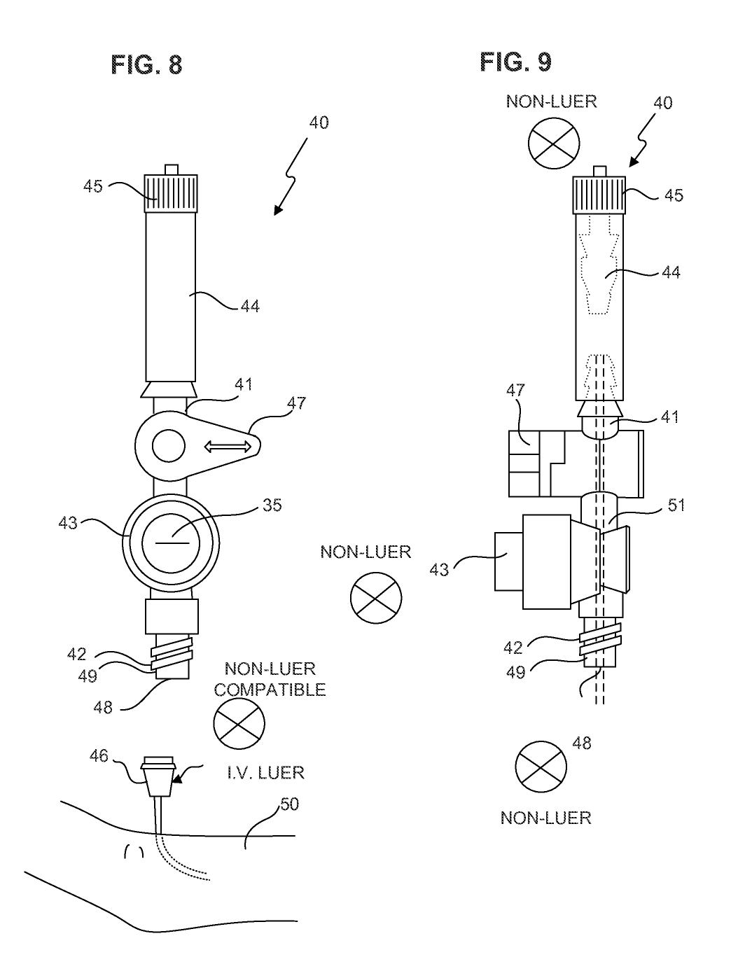

The exemplary embodiments described herein show several non-luer compatible forms, including a female threaded form (e.g., 42 in FIG. 8), a stepped barb form (e.g., 34 in FIG. 7), and male stem form (e.g., 29 in FIG. 5). Other variations are possible and are consistent with the principles of this disclosure.

A clearer partial cross sectional view of non-luer compatible syringe port seal 30 is shown in FIG. 6. The syringe port seal 30 from FIGS. 5 and 6, while similar to the seal shown and described in FIGS. 3 and 4, differs in the upper entrance port configuration 31 wherein the upper entrance opening 32 has been enlarged to 8.76 mm in diameter from its previous 8.0 mm to now be designated as a non-small bore connector port by recently proposed ISO/ANSI 80369-3 standards, which define small bore connectors as having entrance flow paths of less than 8.5 mm. Also, the reconfiguration of opening 32 will now even accept the larger dimensioned catheter tip irrigation syringes from Covidien.

Syringe port seal 30 will accept a catheter tip syringe 16 having a tip 15, whose length and/or diameter are configured in excess of the male luer tip that is in accordance with ISO/ANSI 1986 standards. As used herein, the term “catheter tip syringe” is defined broadly to include any type of oversized syringe tip including oral/enteral purple/orange colored and designated syringes from Becton, Dickinson Co. and Kendall Monoject®.

FIG. 7 is a side view of the exemplary embodiment of connector 23 from FIG. 5 wherein the distal patient access fitting 24 directly attaches or connects to a wide variety of enteral tubes such as gastrostomy, jejunal, Salem Sump®, or Levin tubes 33. This is accomplished by a plurality of stepped barbs 34 molded into fitting 24.

Further, syringe port seal 30 is normally closed to atmosphere by silicone diaphragm slit valve 35 shown in FIG. 8.

In an alternate embodiment, included within injection molded body 38 made from rigid clear polycarbonate is an On-Off fluid controller 36 having built-in fixed stops 37 to place turn handle 39 at either the fluid On or Off position.

From FIG. 7, once triple-ported connector 23 is connected to enteral tubes 33 by distal fitting 24 both normally closed to atmosphere syringe port seal 30 and normally closed off to atmosphere On-Off fluid controller 36 provide a closed system enteral connector 23 preventing any potentially infectious enteral stomach contents from exiting out either syringe port seal 30 or proximal port 28. This protects the clinician from any contact or splash back of enteral stomach contents out any port of connector 23 during patient care operation of connector 23. Likewise, the closed system configuration of both syringe port 30 and fluid controller 36 prevents any outside contamination from entering into enteral tubes 33 to further protect the patient.

FIGS. 8 and 9 depict a second exemplary embodiment of the connector 40 having virtually identical components as shown in FIGS. 6 and 7 with the exception of a molded-in oversized female externally threaded distal patient access fitting 42, whose internal diameter 48 dimensionally exceeds the ISO/ANSI 1986 standards. Connector 40 has a syringe port seal 43, a body portion 41, a proximal fitting 44 and a proximal port 45, and an On-Off fluid controller 47.

Note a distal patient access fitting 42 with an internal diameter 48 of 5.74 mm and an external diameter 49 of 10 mm, which is way oversized in dimension to prevent use, attachment, or mechanical engagement with a much smaller dimensionally configured I.V. luer fitting 46 inserted into a patient arm 50. Further, both the distal fitting 42 and the proximal fitting 44 both have fluid flow paths in excess of 2.4 mm, which exceeds the maximum internal flow path of any luer connector.

FIG. 9 depicts a side view of the second exemplary embodiment of the connector from FIG. 8, which depicts the body portion 41 on connector 40 having a fluid flow path 51.

Accordingly, as shown and described from FIGS. 5-9, both closed system triple-ported connector 23 from FIG. 7 and closed system triple-ported connector 40 from FIG. 8 have respective body portions 38 and 41 having non-luer compatible distal fitting 24 and 42, non-luer compatible proximal fluid delivery device fitting 27 and 44, and non-luer compatible syringe port 30 and 43. All three fittings, distal fitting 24 and 42, proximal fitting 27 and 44, and syringe port 30 and 43, are uniquely individually designed, created, and configured to prevent use, attachment, mechanical engagement, or misconnection with any male or female luer fitting defined by the ISO/ANSI 1986 standards by preventing any mating dimensions, surface to surface mechanical press, and/or interlocking engagement fit, thereby preventing any possible user misconnection between any two of the three fittings and between any fitting on the connector and any luer fitting whether rigid or flexible.

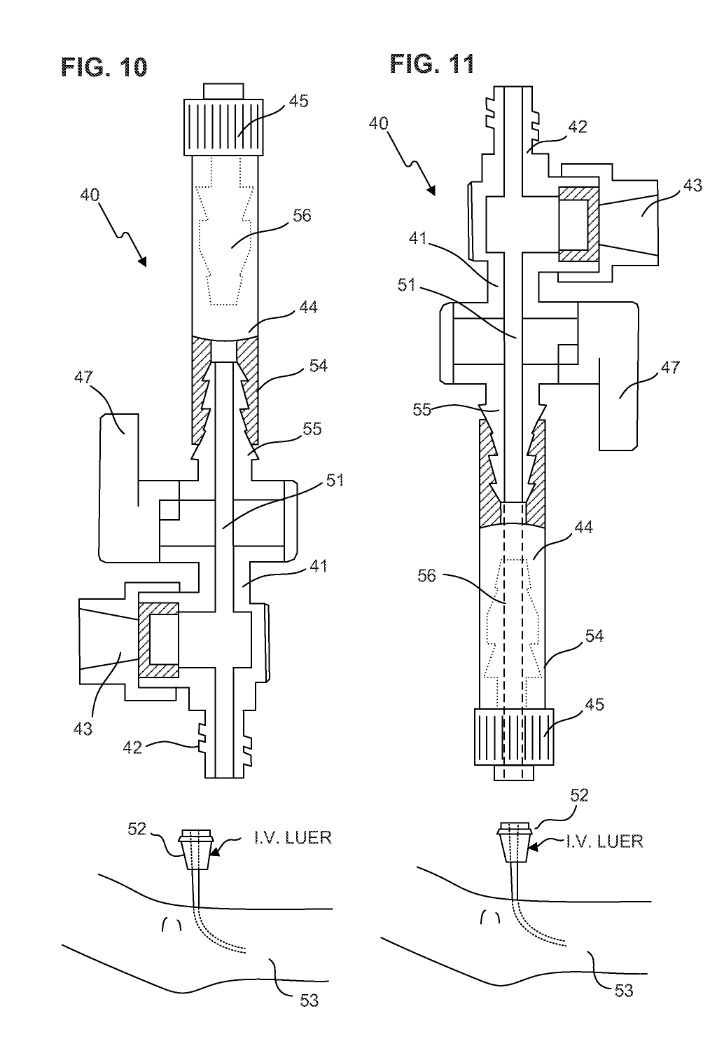

FIGS. 10 and 11 depict a partial cross sectional view of the second exemplary embodiment of the connector 40 from FIGS. 8 and 9. FIGS. 10 and 11 illustrate a fluid flow path 51 within body portion 41 wherein all ports 42, 43, and 44 are all in fluid communication with fluid flow path 51, and both distal and proximal fittings 42 or 45 are configured to prevent use, attachment, or mechanical engagement with any female luer fitting 52 depicted on a patient's arm 53.

FIGS. 10 and 11 also depict an exemplary embodiment of an underlying auxiliary suction line connector 55 molded and built into the body portion 41.

The proximal fitting 44 is comprised of two joined components comprising flexible silicone extruded bushing 54 of approximately Shore A 50 durometer. The proximal port 45 has a plurality of barbed elements 56 to be press fit into the flexible bushing 54. The silicone bushing 54 permits removal of proximal fitting 44 to expose the underlying auxiliary suction line connector 55 part of body portion 41 in case the clinician user wishes to attach the connector 40 to wall suction using built in suction line connector 55 to decompress the patient's stomach or GI tract.

Proximal fitting 44 can be fixedly press fit or removable and replaceable from body portion 41. Many clinicians may prefer that silicone bushing 54 be removable and replaceable especially if fitting 44 were clogged due to lack of flushing or irrigation through fitting 44. As such, the silicone bushing 54 can be packaged separately and marketed as a replaceable component.

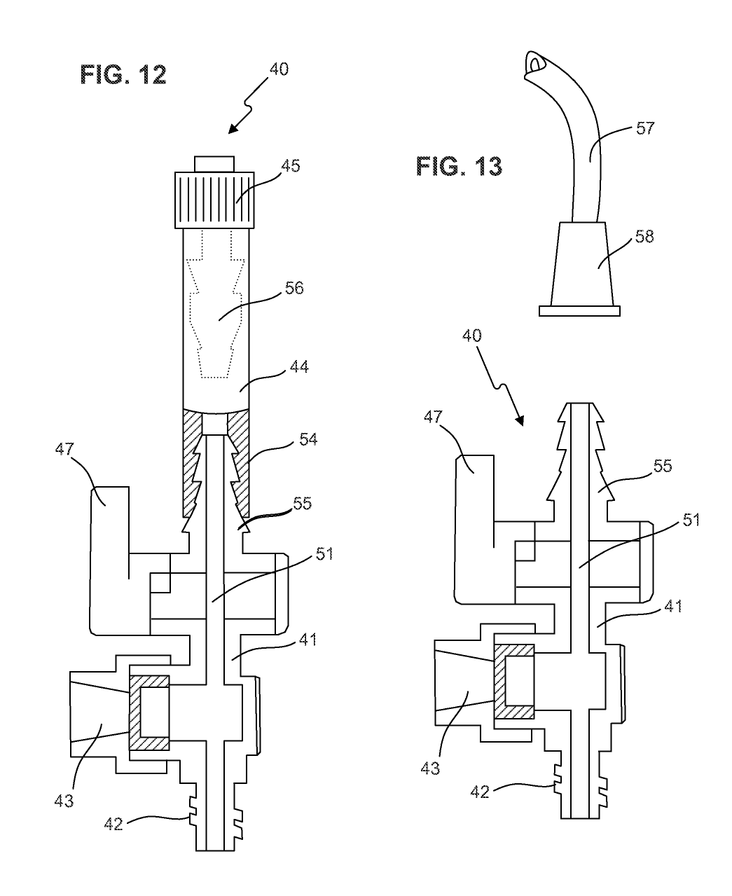

FIGS. 12 and 13 depict the exemplary embodiment of the connector 40 from FIGS. 10 and 11. FIG. 12 depicts connector 40 with proximal fitting 44 press fit into the suction connector 55 and FIG. 13 depicts connector 40 with proximal fitting 44 removed and the rear suction connector 55 easily connected to a suction line 57 having a standard suction fitting 58.

While the barb elements 56 are press fit into the flexible bushing 54 to connect proximal port 45 with silicone bushing 54, it is easily understood that the proximal port 45 could be removable from bushing 54, but that is not the preferred embodiment of a unitized assembly between silicone bushing 54 and proximal port 45.

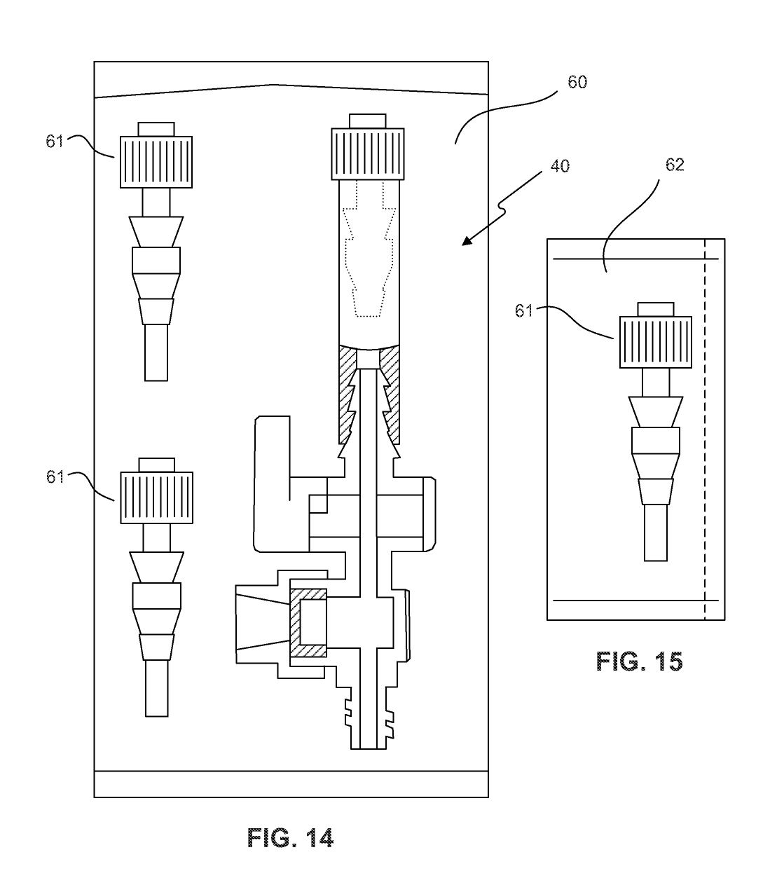

FIG. 14 depicts a preferred exemplary packaging assembly of connector 40 from FIG. 10. The connector 40 is packaged in a non-sterile peel back pouch 60 wherein the package includes two rigid one-piece injection molded polycarbonate adapters 61 which are identical. Separately packaged replacement adapters 61 can be made available in a separate package 62 as shown in FIG. 15.

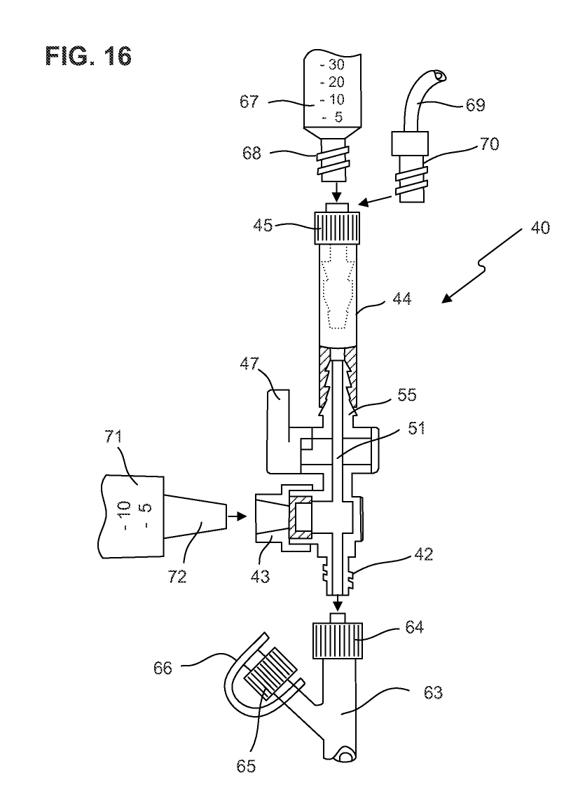

FIG. 16 is a partial cross sectional side view of the exemplary embodiment of the connector 40 from FIG. 10. FIG. 16 depicts how connector 40 can be used with any feeding devices or enteral feeding tubes in accordance to the newly proposed ISO/ANSI 80369-3 standards. For example, FIG. 16 depicts connector 40 utilized to directly connect to all the proposed ISO/ANSI 80369-3 devices. Distal female threaded fitting 42 can directly connect to any future designed feeding tube 63 having male stemmed oversized fluid entry ports 64 or 65 which could utilize a tethered cap 66.

Likewise proximal fluid delivery fitting 44 having male port 45 can directly connect to any oversized future designed or marketed ISO/ANSI 80369-3 compliant syringe 67 having a female threaded tip 68 directly attachable to proximal port 45. In addition, the syringe port seal 43 will only accept catheter tip syringe 71 having a non-luer compatible tip 72.

In summary, connector 40 provides a medical connector wherein all access ports are totally non-luer compatible to prevent use, attachment, or engagement with any I.V. luer fittings or catheters.

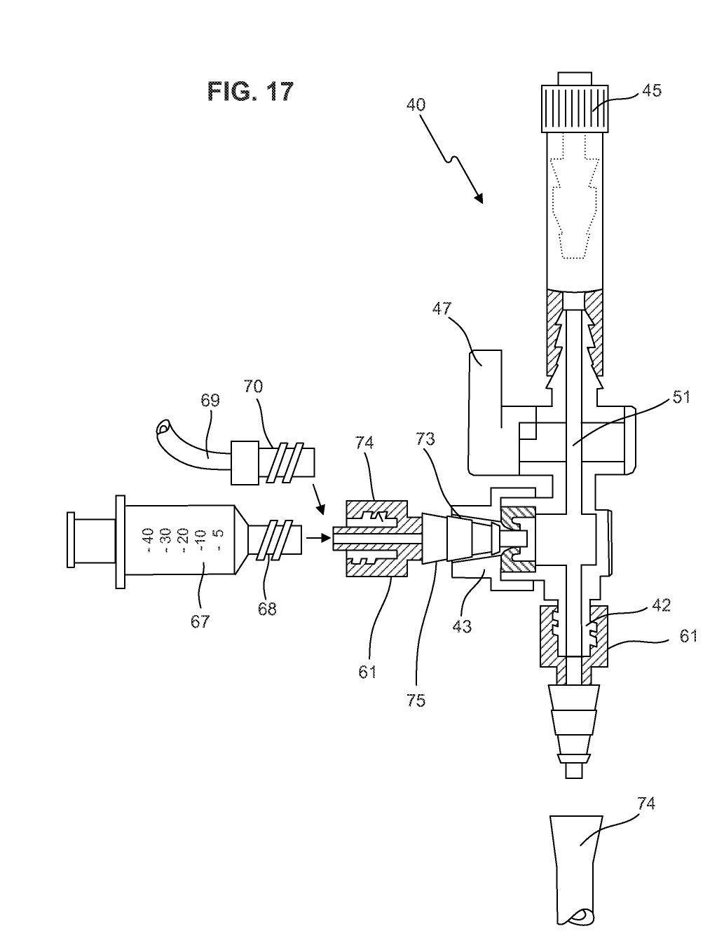

FIG. 17 is a partial cross section side view of the exemplary embodiment of the connector 40 from FIG. 10 depicting all the various non-luer compatible devices that can be used, attached, or mechanically engaged with the connector while utilizing the exemplary adapters depicted in FIG. 14. The versatility and usefulness of these adapters are illustrated and described in FIG. 17.

FIG. 17 depicts how the use of adapters 61 packaged in a kit as shown in FIG. 14 can be used to add versatility to use of the connector 40 while still preserving non-luer compatibility with all the access ports. Advantageously, adapters 61 may be universal so as to be compatible with all three ports of the connectors disclosed herein.

In FIG. 17, first adapter 61 can be used to insert and attach with syringe port seal 43 wherein rigid polycarbonate injection molded adapter 61 has a built-in taper lock wedge portion 75 which taper locks with opening 73 on syringe port seal 43. The taper lock wedge portion 75 may access the valve by having a vertical length great enough to access the valve, in some embodiments, greater than about 0.300 inches. Rearward on adapter 61 is molded in threads 74 which permit direct connection with either fluid delivery device 69 or syringe 67. The syringe port seal 43, which is normally biased to a closed (to atmosphere) position, permits fluid flow from the enteral fluid delivery device 69 or syringe 67 to flow into the fluid flow path 51. Also, any enteral formula delivery set 69 having a matching female threaded tip 70 can directly connect to proximal port 45.

The use of this adapter 61 into syringe port seal 43 now converts the seal 43 into use by any of the future conceived syringe or delivery sets in accordance with ISO/ANSI 80369-3 standards.

Likewise, a second adapter 61 can directly engage by one and one-quarter screw thread with distal fitting 42 to convert distal fitting 42 to be used as a direct connect to any feeding tube 74.

As can be seen, the design, development, and creation of the present disclosure provides for a unique closed system triple-ported connector with non-luer compatible access ports designed and configured for maximum patient safety for present day use as well as anticipation for meeting any future universal enteral feeding device connections as envisioned by the proposed ISO/ANSI 80369-3 standards.

Other embodiments will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical connector preventing use and mechanical engagement with an I.V. luer catheter, the connector comprising:
a body portion having
a distal patient access fitting having a first non-luer compatible form,
a proximal fluid delivery device fitting having a second non-luer compatible form, and
a syringe port incorporating a normally biased closed to atmosphere valve, the syringe port having a third non-luer compatible form, the third non-luer compatible form comprising a vertical length greater than about 0.300 inches from the entrance of the syringe port to the valve,
wherein the distal patient access fitting, the proximal fluid delivery device fitting, and the syringe port are all in fluid communication with a fluid flow path positioned within the body portion.

2. The medical connector of claim 1, wherein the first non-luer compatible form and second non-luer compatible form are the same form, and wherein the form is selected from the group consisting of a female externally threaded form having an internal diameter of greater than about 0.190 inches, a stepped barb form comprising at least a first stepped end barb having a first diameter of greater than about 0.190 inches, and a second stepped barb having a second diameter greater than the first diameter, and a male stem form having a diameter of greater than about 0.190 inches.

3. The medical connector of claim 1, wherein the first non-luer compatible form and second non-luer compatible form are different forms, and wherein the different forms are selected from the group consisting of a female externally threaded form having an internal diameter of greater than about 0.190 inches, a stepped barb form comprising at least a first stepped end barb having a first diameter of greater than about 0.190 inches, and a second stepped barb having a second diameter greater than the first diameter, and a male stem form having a tapered outside diameter of greater than about 0.190 inches.

4. The medical connector of claim 1, further comprising an adapter engaged with the syringe port, wherein the adapter mechanically engages with an enteral fluid delivery device.

5. The medical connector of claim 4, wherein the adapter accesses the normally biased closed to atmosphere valve, causing the valve to be in an open position permitting fluid flow from the enteral fluid delivery device into the fluid flow path positioned within the body portion.

6. The connector of claim 1 wherein the body portion further comprises an On-Off fluid flow controller disposed between the valve and the proximal fluid delivery device fitting.

7. The connector of claim 6, wherein the On-Off fluid flow controller is a manually operated turn handle.

8. The connector of claim 7, wherein the turn handle has fixed stop positions for both the on and off fluid flow positions.

9. The connector of claim 1, further comprising an adapter attached to the distal patient access fitting, permitting connection of the device to an enteral feeding tube.

10. The connector of claim 9, wherein the adapter has a non-luer compatible form comprising a male stem having a tapered outside diameter of greater than about 0.190 inches.

11. The connector of claim 1, wherein the proximal fluid delivery device fitting connects to an enteral feeding device.

12. The connector of claim 1, wherein the connector is an enteral formula delivery connector.

13. The connector of claim 1, wherein the distal patient access fitting has a flow path internal diameter dimensionally in excess of about 2.4 mm.

14. The connector of claim 1, wherein the proximal fluid delivery device fitting has a flow path internal diameter dimensionally in excess of about 2.4 mm.

15. The connector of claim 1, wherein the proximal fluid delivery device fitting is removable from the body portion.

16. The connector of claim 14, wherein the removal of the proximal fluid delivery device fitting exposes an underlying auxiliary suction line connector part of the body portion.

17. The connector of claim 1, wherein a fluid flow path of the distal patient access fitting and a fluid flow path of the proximal fluid delivery device fitting have diameters exceeding about 2.4 mm.

18. The connector of claim 1, wherein the valved syringe access port accepts a catheter tip syringe having a length in excess of about 0.300 inches.

19. A medical connector kit preventing use and mechanical engagement with an I.V. luer catheter, the connector kit comprising:
a medical connector comprising a body portion having
a distal patient access fitting having a first non-luer compatible form,
a proximal fluid delivery device fitting having a second non-luer compatible form, and
a syringe port incorporating a normally biased closed to atmosphere valve, the syringe port having a third non-luer compatible form, the third non-luer compatible form comprising a vertical length greater than about 0.300 inches from the entrance of the syringe port to the valve,
wherein the distal patient access fitting, the proximal fluid delivery device fitting, and the syringe port are all in fluid communication with a fluid flow path positioned within the body portion; and
at least one adapter, the at least one adapter comprising a proximal portion comprising a taper lock wedge, the taper lock wedge having a minimum diameter in excess of about 0.190 inches, and a rearward portion comprising molded-in threads.

20. The medical connector kit of claim 19, wherein the taper lock wedge of the proximal portion of the adapter has a vertical length greater than about 0.300 inches.
